(19) United States
(12) Patent Application Publication
(10) Pub. No.: US 2024/0052303 A1
(43) Pub. Date: Feb. 15, 2024

(54) METHODS OF EMBRYO TWINNING

(71) Applicant: NBRYO PTY LTD, Yeerongpilly, Queensland (AU)

(72) Inventors: Nicholas CAMERON, Tabragalba, Queensland (AU); Danielle AZOULAY, Leichhardt, New South Wales (AU)

(21) Appl. No.: 18/018,831

(22) PCT Filed: Jul. 30, 2021
(86) PCT No.: PCT/AU2021/050836

§ 371 Date: Jan. 30, 2023

(30) Foreign Application Priority Data
Jul. 31, 2020 (AU) 2020902691

(51) Int. Cl. C12N 5/073; C12N 15/877

(52) U.S. Cl. CPC C12N 5/0604; C12N 15/8771; C12N 2509/00; C12N 2500/32; C12N 2500/34; C12N 2500/35

(57) ABSTRACT

The present disclosure relates generally to methods of producing multiple embryos from one or more donor embryos by serial multiplication, for example, by performing 3 or more rounds of multiplication, as well as the use of such methods in animal breeding. The present disclosure also relates to methods of producing multiple monozygotic embryos from a donor embryo which comprises embryonic cells which are developmentally equivalent to embryonic cells from a 16-cell embryo or a pre-compacted morula.

### RELATED APPLICATION DATA

This application claims the right of priority to Australian Provisional Application No. 2020902691, filed 31 Jul. 2020, the complete contents of which is incorporated by reference herein in its entirety.

### TECHNICAL FIELD

The present disclosure relates generally to methods of producing multiple embryos from one or more donor embryos by serial multiplication cycles, for example, by performing three or more rounds of multiplication, as well as the use of such methods in animal breeding. The present disclosure also relates to methods of producing multiple monozygotic embryos from a donor embryo that comprises embryonic cells that are developmentally equivalent to embryonic cells from a 16-cell embryo or a pre-compacted morula.

### BACKGROUND

Assisted reproductive technologies (ART) have made tremendous advances, particularly during the past few decades. Artificial insemination (AI) remains the most (cost) effective method for achieving genetic gain in cattle populations and is widely used in the dairy industry. In this regard, the global market remains strong for frozen semen and embryos, with millions of cattle bred by AI, and more than a million embryos transferred annually worldwide. Most of the top sires in the dairy industry that provide semen for AI are derived from embryo transfer (ET), and improvements in methods of controlling the oestrous cycle and ovulation have resulted in more effective programs for AI, superovulation of donor cows, and the management of ET recipients. Notwithstanding these advanced in ART, uptake by producers of reproductive technologies like multiple ovulation and embryo transfer (MOET) remains limited owing to the expense associated with the production of each embryo. Unlike conventional AI, MOET is therefore unlikely to be used by producers as a conventional reproductive method.

More recently, approaches for producing genetically identical monozygotic twins by embryo bisection, as well as from blastomeres separated from cleavage stage embryos, have been reported. Whilst this new addition to the ART “toolbox” is exciting and would enable producers' to more effectively capture and select for female (dam) genetics (in addition to sire genetics), the widespread adoption of embryo twinning, like other ET- and IVF-based approaches, at a commercial level is likely to be hampered by the prohibitive cost to the producer, as well as by the challenges associated with scaling of the technology.

Accordingly, there is a need for improved approaches for embryo multiplication to address one or more of these limitations and assist with uptake by industry.

### SUMMARY

The present disclosure is broadly directed to methods of producing multiple embryos from one or more donor embryos. In this regard, the inventors have shown for the first time that bovine donor embryos comprising at least 2 embryonic cells can be multiplied by ≥3 serial rounds of multiplication prior to expansion of the resultant embryos to blastocyst stage. In doing so, the inventors have shown that the efficiency of blastocyst recovery from the initial donor embryos using the serial multiplication method described herein is significantly higher (e.g., as much as 6-fold higher) than if the initial intact donor embryos were simply cultured straight through to blastocysts.

The inventors have also shown for the first time that bovine embryos comprising one or more embryonic cells that are developmentally equivalent to embryonic cells from a 16-cell embryo or a pre-compacted morula, can be used as donor embryos to produce multiple monozygotic embryos, including when serial multiplication steps as described herein are employed to further multiply the embryos. Starting with these more developed donor embryos, which frequently comprise a heterogenous mixture of blastomeres developmentally equivalent to cell from 8-, 16-, 32- and 64-cell embryos, the inventors have shown that the efficiency of blastocyst recovery using the multiplication method described herein is significantly higher (e.g., as much as 10-fold higher when serial multiplication is employed) than if the initial intact donor embryos were simply cultured to straight through to blastocysts.

In one example, the disclosure provides a method of multiplying one or more donor embryos, said method comprising:
(i) obtaining one or more donor embryo comprising at least two embryonic cells;
(ii) separating one or more of the embryonic cells from the one or more donor embryos;
(iii) expanding the embryonic cells in vitro under conditions suitable to produce a plurality of embryos, each comprising at least two embryonic cells;
(iv) isolating one or more of the plurality of embryos produced at (iii) to be used as donor embryos in subsequent multiplications; and
(v) repeating steps (i)-(iv) ‘n’ times, wherein ‘n’ is ≥3.

In another example, the disclosure provides a method of multiplying a donor embryo, said method comprising:
(i) obtaining a donor embryo comprising one or more embryonic cells that are developmentally equivalent to embryonic cells from a 16-cell embryo or a pre-compacted morula;
(ii) separating one or more of the embryonic cells from the donor embryo;
(iii) expanding the embryonic cells in vitro under conditions suitable to produce a plurality of monozygotic embryos from the donor embryo; and
(iv) culturing the plurality of monozygotic embryos under conditions suitable to produce a plurality of monozygotic blastocysts.

Prior to the step of culturing the plurality of monozygotic embryos to produce the plurality of blastocysts, the method may further comprises the steps of:
(i) isolating one or more of the plurality of monozygotic embryos produced to be used as donor embryos in subsequent multiplications, wherein each donor embryo isolated for subsequent multiplications comprises at least two embryonic cells;
(ii) separating one or more of the embryonic cells from the one or more donor embryos;
(iii) expanding the embryonic cells in vitro under conditions suitable to produce a plurality of embryos, each comprising at least two embryonic cells;
(iv) isolating one or more of the plurality of embryos produced at (iii) to be used as donor embryos in subsequent multiplications; and
(v) repeating steps (i)-(iv) ‘n’ times before culturing the plurality of embryos under conditions suitable to produce a plurality of blastocysts.

In one example, separation of the one or more embryonic cells from each donor embryo is achieved by splitting the donor embryo into two or more portions, each portion (or demi-embryo) comprising one or more embryonic cells. In one example, at least one donor embryo may be split into two portions. In one example, at least one donor embryo may be split into three portions. In one example, at least one donor embryo may be split into four portions. In one example, at least one donor embryo may be split into five or more portions.

In each of the foregoing examples, the donor embryos may be split or cut using a microsurgical instrument. For example, the donor embryos may be split or cut by microdissection using a blade (e.g., a scalpel blade or portion thereof), a fine glass needle or a laser e.g., laser-assisted biopsy. In other examples, the donor embryos may be split or cut by nano-dissection (e.g., using femtosecond laser pulse or atomic force microscopy (AFM) with a nano-scalpel).

In another example, separation of the one or more embryonic cells from each donor embryo is achieved by disrupting the zona pellucida (ZP), and isolating the one or more of the embryonic cells from the donor embryo. This is referred to herein as “unzipping” or the “unzip method”. In accordance with this example, the ZP may be disrupted and one or more of the embryonic cells isolated from the one or more donor embryos. In one example, the ZP is disrupted enzymatically or mechanically. For example, the ZP may be disrupted enzymatically or mechanically, and a micropipette used to aspirate the one or more of the embryonic cells from the one or more donor embryos, thereby isolating the embryonic cells.

Unless otherwise stated, ‘n’ is ≥1. For example, ‘n’ may be ≥2. For example, ‘n’ may be ≥3. In accordance with examples in which ‘n’ is ≥3, ‘n’ may be ≥4. For example, ‘n’ may be ≥5. For example, ‘n’ may be equal to 6, or 7 or 8 or 9 or 10 or more.

In one example, 16 or more monozygotic embryos are produced by the method of the disclosure. In one example, 32 or more monozygotic embryos are produced by the method of the disclosure. In one example, 64 or more monozygotic embryos are produced by the method of the disclosure. In one example, 128 or more monozygotic embryos are produced by the method of the disclosure. In one example, 256 or more monozygotic embryos are produced by the method of the disclosure. In one example, 512 or more monozygotic embryos are produced by the method of the disclosure.

In one example, the embryonic cells or embryos comprising same may be cultured in the presence of one or more factors capable of promoting embryogenesis. For examples, the embryonic cells may be cultured in the presence of one or more factors capable of promoting embryogenesis in order to form and expand the monozygotic embryos e.g., for harvest.

In another example, the embryonic cells or embryos comprising same may be cultured in the presence of one or more factors capable of promoting totipotency and/or inhibiting or preventing embryogenesis. For example, the embryonic cells or embryos comprising same may be cultured in the presence of one or more factors capable of promoting clearance of maternal mRNAs.

Unless otherwise stated, the or each donor embryo comprises about 2 to about 300 embryonic cells e.g., about 2 to about 256 embryonic cells or about 2 to about 64 embryonic cells, provided that compaction has not yet occurred. For example, the or each donor embryo may comprise about 100 to about 256 embryonic cells, provided that compaction has not yet occurred. For example, the or each donor embryo may comprise about 64 to about 128 embryonic cells, provided that compaction has not yet occurred. For example, the or each donor embryo may comprise about 32 to about 64 embryonic cells, provided that compaction has not yet occurred. For example, the or each donor embryo may comprise about 16 to about 32 embryonic cells, provided that compaction has not yet occurred. For example, the or each donor embryo may comprise about 2 to about 32 embryonic cells, provided that compaction has not yet occurred. For example, the or each donor embryo may comprise about 2 to about 16 embryonic cells, provided that compaction has not yet occurred. For example, the or each donor embryo may comprise about 2 to about 8 embryonic cells.

In accordance with an example in which the donor embryo comprises one or more embryonic cells which are developmentally equivalent to embryonic cells from a 16-cell embryo or a pre-compacted morula, the donor embryo may comprise about 12 to about 32 embryonic cells which have not yet compacted. In accordance with this example, the donor embryo may comprise one or more embryonic cells which are developmentally equivalent to embryonic cells from a 8-cell embryo, one or more embryonic cells which are developmentally equivalent to embryonic cells from a 16-cell embryo, one or more embryonic cells which are developmentally equivalent to embryonic cells from a 32-cell embryo, and/or one or more embryonic cells which are developmentally equivalent to embryonic cells from a 64-cell embryo.

In each of the foregoing examples, the donor embryos may be obtained from a vertebrate animal.

In one example, the vertebrate animal may be a mammalian species.

In one example, the mammalian species may be a livestock species. For example, the livestock species may be a bovine species. For example, the livestock species may be a ovine species (i.e., sheep). For example, the livestock species may be a porcine species (i.e., pig). For example, the livestock species may be an equine species (i.e., horse). For example, the livestock species may be a caprine species (i.e., goat). For example, the livestock species may be a cervid species (i.e., deer). For example, the livestock species may be a camelid species (e.g., camel or alpaca).

In some examples, one or more of the donor embryo obtained at step (i) is/are produced by in vivo fertilisation. In other examples, one or more of the donor embryo obtained at step (i) is/are produced by in vitro fertilisation (IVF).

In some examples, one or more of the donor embryos obtained at step (i) are fresh. In other examples, one or more of the donor embryos obtained at step (i) have been cryopreserved. For example, the donor embryos may be thawed.

In each of the forgoing examples, the method may further comprise selecting one or more of the donor embryos obtained at step (i) on the basis of one or more genetic screening criteria, genetic diagnoses and/or one or more morphological criteria. For example, the selection step may be performed prior to step (i).

In one example, the genetic screening criteria may be determined by screening the one or more donor embryos for the presence or absence of one or more genetic markers (e.g., SNP alleles or haplotype) associated with a trait of interest. In one example, the trait of interest is selected from a phenotypic production trait, drug resistance, susceptibility to pests and/or parasites, and sex (i.e., determining whether the embryo is male or female).

In one example, one or more of the donor embryos may be selected on the basis of a genetic diagnosis for one or more conditions, diseases or predisposition thereto.

In one example, one or more of the donor embryos may be selected on the basis of one or more morphological characteristics which is indicative of embryo health.

In each of the foregoing examples, one or more of the donor embryos may be genetically modified. For example, one or more of the donor embryos may be genetically modified by introducing an exogenous nucleic acid to the genome of the embryonic cells comprised therein. For example, one or more of the donor embryos may be genetically modified by editing the genome of the embryonic cells comprised therein.

In one example, one or more of the donor embryo comprises a unique genetic tag or nucleic acid identifier for traceability of the embryos produced therefrom and/or animals produced from said embryos. For example, the unique genetic tag or nucleic acid identifier may be introduced using genetic modification.

In each of the foregoing example, the method comprises expanding the plurality of embryos in vitro to form blastocysts. For example, the method may comprises expanding the embryos in vitro to form mature blastocysts which are ready for implantation.

The method may further comprise harvesting the plurality of embryos produced by the method. For example, the method may comprise harvesting the embryos once they mature to the blastocyst stage.

In some examples, one or more of the harvested embryos are stored in an embryo holding media. For example, the one or more of the harvested embryos may be stored at about 4° C.

In some examples, the one or more of the harvested embryos are cryopreserved. Cryopreserved embryos may be stored at about −180° C. to about −196° C. For example, the cryopreserved embryos may be stored in liquid nitrogen at about −196° C.

In some example, the method of the disclosure further comprises transferring one or more of the embryos produced by the method to the oviduct(s) of one of more recipient females.

The present disclosure also provides one or more embryos produced by the method of the disclosure. In one example, the one or more embryos may be provided in embryo storage or transfer media at about 4° C. In another example, the one or more embryos may be cryopreserved.

In one example, the embryo is from a mammalian species (e.g., a non-mammalian species). In one example, the non-human mammalian species is a livestock species. For example, the livestock species may be a bovine species. For example, the livestock species may be a ovine species (i.e., sheep). For example, the livestock species may be a porcine species (i.e., pig). For example, the livestock species may be an equine species (i.e., horse). For example, the livestock species may be a caprine species (i.e., goat). For example, the livestock species may be a cervid species (i.e., deer). For example, the livestock species may be a camelid species (e.g., camel or alpaca).

The present disclosure also provides a method of breeding an animal, comprising:
(i) transferring one or more of the embryos produced by the method of the disclosure to the oviduct(s) of one of more recipient females to establish a pregnancy;
(ii) producing the animal from the pregnant recipient female by parturition.

In one example, the animal is a vertebrate animal. For example, the vertebrate animal may be a mammal, an amphibian, a reptile, a fish or a bird.

In one particular example, the animal is a mammal e.g., a non-human mammal. Exemplary non-human mammals which may be produced using the method include livestock species (e.g., cattle, buffalo, pigs, sheep, goats, camelid, deer, horses etc.), companion animals (e.g., dogs, cats etc.), laboratory animals (e.g., rats, mice, hamsters, guinea pigs, rabbits, etc.)., non-human primates (macaque and marmoset etc.) and wildlife species (e.g., marsupials, cats, rhino, giant panda etc.). In one particular example, the method of the disclosure may be used to breed cattle. In another example, the method of the disclosure may be used to breed sheep. In another example, the method of the disclosure may be used to breed pigs. In another example, the method of the disclosure may be used to breed goats. In another example, the method of the disclosure may be used to breed horses.

### BRIEF DESCRIPTION OF THE DRAWING

FIG. 1 is a schematic representation of the classification scheme for blastomeres and developing embryos during 4 rounds of unzipping from the 2-cell stage embryos. The left hand side represents normal development of preimplantation conceptus development from the zygote stage to the blastocyst stage. During the unzipping procedure, the Zona pellucida (ZP) (grey band surrounding the conceptus) is removed to separate individual blastomeres within the conceptus (number of serial splits, n=1). Individual blastomeres isolated from the 2-cell conceptus are referred as 1:2. These blastomeres are then allowed to develop to form pairs, named 2:4. Subsequent to cleavage, these 2:4 blastomeres can be taken to another round of unzipping (number of serial splits, n=2), where the blastomeres will be separated to 1:4. The process is repeated for two further rounds of serial splitting, n=3 and n=4 respectively. As these blastomeres develop, they transition to the subsequent equivalent preimplantation conceptus stage and therefore the denominator changes accordingly. After the 1:16 stage, blastomeres can compact, cavitate to then form a blastocyst equivalent.

### DETAILED DESCRIPTION

### General Techniques and Definitions

Unless specifically defined otherwise, all technical and scientific terms used herein shall be taken to have the same meaning as commonly understood by one of ordinary skill in the art (e.g. in animal nutrition, feed formulation, microbiology, livestock management).

As used herein, the singular forms of “a”, “and” and “the” include plural forms of these words, unless the context clearly dictates otherwise.

The term “and/or”, e.g., “X and/or Y” shall be understood to mean either “X and Y” or “X or Y” and shall be taken to provide explicit support for both meanings or for either meaning.

Throughout this specification, the word “comprise” or variations such as “comprises” or “comprising” will be understood to imply the inclusion of a stated element, integer or step, or group of elements, integers or steps, but not the exclusion of any other element, integer or step, or group of elements, integers or steps.

The term “about” is used herein to mean approximately. When the term “about” is used in conjunction with a numerical range, it modifies that range by extending the boundaries above and below the recited numerical values. In general, the term “about” is used herein to modify a numerical value above and below the stated value by 10%, up or down (higher or lower).

Those skilled in the art will appreciate that the present disclosure is susceptible to variations and modifications other than those specifically described. It is to be understood that the disclosure includes all such variations and modifications. The disclosure also includes all of the steps, features, compositions and compounds referred to or indicated in this specification, individually or collectively, and any and all combinations of any two or more of said steps or features. Thus, each feature of any particular aspect or embodiment of the present disclosure may be applied mutatis mutandis to any other aspect or embodiment of the present disclosure.

The present disclosure is not to be limited in scope by the specific embodiments described herein, which are intended for the purpose of exemplification only. Functionally equivalent products, compositions and methods are clearly within the scope of the disclosure, as described herein.

Throughout this specification, unless specifically stated otherwise or the context requires otherwise, reference to a single step, composition of matter, group of steps or group of compositions of matter shall be taken to encompass one and a plurality (i.e. one or more) of those steps, compositions of matter, groups of steps or group of compositions of matter.

### Specific Definitions

The term “embryo’ is used herein to refer to the zygote that is formed when two haploid gametic cells (e.g., an unfertilized oocyte and a sperm cell) unite to form a diploid totipotent cell (e.g., a fertilized ovum), as well as to the embryo that results from the subsequent cell divisions (i.e. embryonic cleavage), including the morula stage (i.e. when the inner cell mass has compacted) and blastocyst stage with differentiated trophoectoderm and inner cell mass.

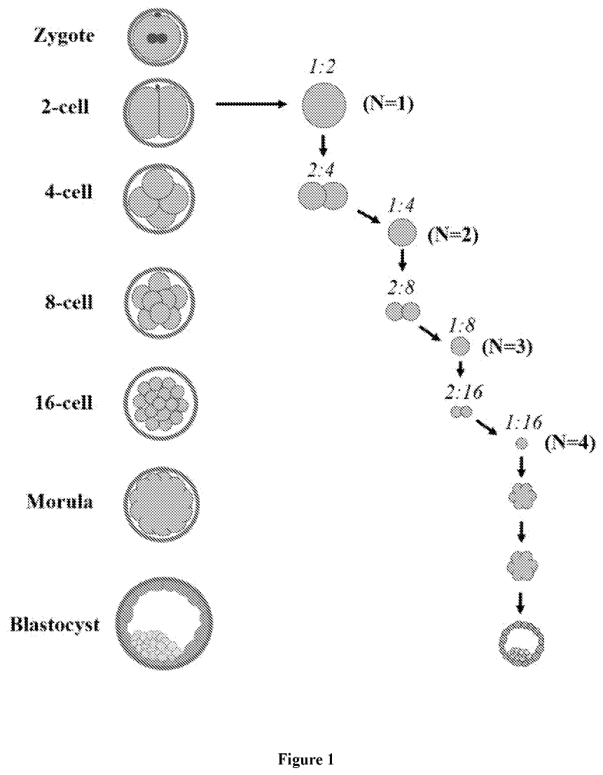

As used herein, the term “morula” refers to a stage of embryonic development. The morula is an early stage embryo that consists of a ball of cells (called blastomeres) contained within a glycoprotein membrane called a zona pellucida. The morula is produced from the single-celled zygote through a series of cleavage events (which is illustrated in FIG. 1 for bovine). A key event prior to morula formation is “compaction”, where the embryo containing about 8-32 cells (depending on species) undergoes changes in cell morphology and cell-cell adhesion that initiates the formation of this solid ball of cells. A “morula” typically comprises around 16-32 cells (depending on species), and resembles mulberry, hence the name morula (Latin, moms: mulberry). In the context of bovine species, the process of compaction typically takes place after the 16-cell stage and the developing embryo reaches the early morula stage at the 32-cell stage, at which time cell-to-cell adhesion between the embryonic cells (or blastomeres) is progressed and the embryo comprises an compacted inner cell mass (ICM).

Through a process involving cellular differentiation and cavitation, the morula gives rise to a blastocyst. As used herein, the term “blastocyst” shall be understood to refer to an embryo which possesses an inner cell mass (ICM), or embryoblast comprising totipotent embryonic stem cells, and an outer layer of cells, or trophoblast, which later forms the placenta. The trophoblast surrounds the inner cell mass and a fluid-filled blastocyst cavity known as the blastocoel. A blastocyst typically comprises between 70-300 embryonic cells (which may vary depending on species and maturity of the embryo). In some examples, a blastocyst may comprise about 64 to about 128 cells. In some examples, a blastocyst may comprise between about 128 to about 256 cells. In some examples, a blastocyst may comprise between 150-256 cells. In some examples, a blastocyst may comprise between about 256 cells.

As used herein, the terms “embryonic cell” or “embryonic cells” is intended to encompass all totipotent cells within developing embryo from the zygote to the blastocyst stage. For example, embryonic cells obtained from within the developing embryo from zygote to morula stage (also referred to as “blastomeres”) are totipotent embryonic cells. Likewise, embryonic cells obtained from the inner cell mass of a blastocyst may be totipotent.

As used herein, the term “totipotent” is used to describe a cell that is capable of giving rise to any cell type. For example, in the context of embryonic cells, a “totipotent” cell is one that can give rise to all of the cell types in an embryo, and ultimately differentiate in any one of the specialised cells required for different tissues in the body (e.g., skin, bone, marrow and muscle etc.). The term “totipotent” is to be distinguished from the term “pluripotent”, the latter referring to cells that differentiate into specific subpopulations of cells within a developing cell mass but which may not give rise to any and all cell types.

As used herein, the term “monozygotic embryos” shall be understood to mean two or more embryos formed or derived from a single zygote.

The term “demi-embryo” as used herein shall be understood to mean a portion of an embryo after it has been split or cut. For example, an embryo that is bisected will produce two demi-embryos, each comprising embryonic cells. Likewise, an embryo that has been cut into three portions, each comprising embryonic cells, will give rise to three demi-embryos.

As used herein, the term “animal” shall be understood to include all vertebrate animals, such as mammals (i.e., non-human mammals), amphibians, reptile, fish and birds. In one example, the animal is a mammal. Exemplary mammals for which the method of the disclosure may be useful include livestock (e.g., cattle, buffalo, pigs, sheep, goats, camelid, deer, horses etc.), companion animals (e.g., dogs, cats etc.), laboratory animals (e.g., rats, mice, hamsters, guinea pigs, rabbits, etc.), non-human primates (macaque and marmoset etc.) and wildlife species (e.g., marsupials, large cats, rhino, giant panda etc.). In one particular example, the method of the disclosure may be useful in cattle (i.e., bovine species).

### Twinning Methods

The present disclosure is directed generally to methods of multiplying embryos, also referred to herein as “twinning”, and in particular, to methods capable of producing a plurality of embryos from one or more initial donor embryos. Several approaches or ‘twinning techniques’ are described herein for producing a plurality of embryos from one or more initial donor embryos.

In one example, a method of the disclosure possesses the following general method steps:
(i) obtaining one or more donor embryo comprising at least two embryonic cells;
(ii) separating one or more of the embryonic cells from the one or more donor embryos;
(iii) expanding the embryonic cells in vitro under conditions suitable to produce a plurality of embryos, each comprising at least two embryonic cells;
(iv) isolating one or more of the plurality of embryos produced at (iii) to be used as donor embryos in subsequent multiplications; and
(v) repeating steps (i)-(iv) ‘n’ times, wherein ‘n’ is ≥3.

In another example, a method of the disclosure that multiplies donor embryos using the ‘twinning techniques’ described herein possesses the following general method steps:
(i) obtaining a donor embryo comprising one or more embryonic cells that are developmentally equivalent to embryonic cells from a 16-cell embryo or a pre-compacted morula;
(ii) separating one or more of the embryonic cells from the donor embryo;
(iii) expanding the embryonic cells in vitro under conditions suitable to produce a plurality of monozygotic embryos from the donor embryo; and
(iv) culturing the plurality of monozygotic embryos under conditions suitable to produce a plurality of monozygotic blastocysts.

In the latter method, prior to the step of culturing the plurality of monozygotic embryos to produce the plurality of blastocysts, the method may further comprises the steps of:
(i) isolating one or more of the plurality of monozygotic embryos produced to be used as donor embryos in subsequent multiplications, wherein each donor embryo isolated for subsequent multiplications comprises at least two embryonic cells;
(ii) separating one or more of the embryonic cells from the one or more donor embryos;
(iii) expanding the embryonic cells in vitro under conditions suitable to produce a plurality of embryos, each comprising at least two embryonic cells;
(iv) isolating one or more of the plurality of embryos produced at (iii) to be used as donor embryos in subsequent multiplications; and
(v) repeating steps (i)-(iv) ‘n’ times before culturing the plurality of embryos under conditions suitable to produce a plurality of blastocysts.

As described herein, steps (i)-(iv) of the method may be repeated ‘n’ times in order to produce a plurality of embryos from the original donor embryos. Depending on (1) the number of embryonic cells in the initial donor embryos, (2) the technique(s) used to separate the embryonic cells therefrom and (3) unless stated otherwise, the number of repetitions of steps (i)-(iv) to be performed i.e., ‘n’, may vary. In this regard, and unless stated otherwise, ‘n’ may be ≥1, e.g., 1, or 2, or 3, or 4, or 5, or 6, or 7, or 8, or 9 or 10 or more. In accordance with examples where the method requires that ‘n’ is ≥3, ‘n’ may be 3, or 4, or 5, or 6, or 7, or 8, or 9 or 10 or more.

In one embodiment, the ‘twinning technique’ employed is designated “the cutting method” or “cut method”. In accordance with this embodiment, the one or more initial donor embryos at step (i) are cut (or split) into two portions (e.g., of approximately equal proportions) comprising one or more embryonic cells. Both of the demi-embryos are then expanded in vitro to produce two monozygotic embryos as set out in steps (i)-(iii) above. The process in steps (i)-(iii) is then repeated in step (iv) using the newly multiplied embryos as donor embryos. As indicated in step (v), the process can be repeated ‘n’ number of times (each referred to as a cycle), using the embryos produced from the previous cycle as donors embryos for the subsequent cycle. In this way, each new cycle of multiplication using the cutting method is capable of doubling the number of monozygotic embryos produced relative to the previous cycle. The number of cycles to be performed using the cutting method will depend on a number of factors (e.g., the number of embryos to be produced, the number of starting donor embryos, the developmental stage of the donor embryos, whether or not any embryos are harvested from the method during intervening cycles etc.), and may therefore be varied.

In one example, the one or more initial donor embryos each comprise at least 2 embryonic cells and the minimum number of cycles to be performed using the cutting method may be three i.e., n≥3.

In another example, the one or more initial donor embryos each comprise one or more embryonic cells that are developmentally equivalent to embryonic cells from a 16-cell embryo or a pre-compacted morula. In accordance with this example, the minimum number of cycles to be performed using the cutting method may be one i.e., n≥1.

In yet another example, the initial donor embryo is a 4-cell embryo and the desired outcome is the production of at least 16 monozygotic embryos. In accordance with this example, a minimum of four cycles (i.e., n≥4) will be necessary using the “cutting method” alone in order to produce at least 16 monozygotic embryos from an initial donor embryo. However, the cutting method may comprise at least five cycles (i.e., n equals 5) or at least six cycles (i.e., n equals 6), or at least seven cycles (i.e., n equals 7), and so on. In this regard, a skilled person will appreciate that the number of cycles to be performed using the cutting method may simply be varied (e.g., increased) based on the number of embryos to be produced from an initial donor embryo and the efficiency of embryo recovery following each cycle.

In another embodiment, the ‘twinning technique’ employed is designated “the cookie cutter method”. In accordance with this embodiment, the one or more initial donor embryos at step (i) are cut (or split) into three, four, five or more portions (e.g., of approximately equal proportions) each comprising one or more embryonic cells, which are then expanded in vitro to produce three or more monozygotic embryos according to steps (i)-(iii) above. The process is then repeated in step (iv) using the newly produced (or ‘twinned’) embryos as donor embryos. As indicated in step (v), the process can be repeated ‘n’ number of times (each referred to as a cycle), using the embryos produced from the previous cycle as donors embryos for the subsequent cycle. The number of cycles to be performed using the ‘cookie cutter’ method will depend on the number of factors, including: the number of embryos to be produced, the number of portions into which the donor embryos are cut in each of the respective cycles (which may be three or more portions and which may vary between cycles), the number of starting donor embryos, the developmental stage of the donor embryos, whether or not any embryos are harvested from the method during intervening cycles etc. Thus, the number of cycles may be varied.

In one example, the one or more initial donor embryos each comprise at least 4 embryonic cells and the minimum number of cycles to be performed using the cookie cutter method may be three i.e., n≥3.

In another example, the one or more initial donor embryos each comprise one or more embryonic cells that are developmentally equivalent to embryonic cells from a 16-cell embryo or a pre-compacted morula. In accordance with this example, the minimum number of cycles to be performed using the cookie cutter may be one i.e., n≥1.

In yet another example, an initial donor embryo is a 4-cell embryo and the desired outcome is the production of at least 16 monozygotic embryos. In accordance with this example, and assuming the donor embryo is cut into three portions in each cycle, the “cookie cutter” method may comprise a minimum of at least three cycles (i.e., n≥3) in order to produce at least 16 monozygotic embryos from an initial donor embryo. However, the cookie cutter method may comprise at least four cycles (i.e., n equals 4), or at least five cycles (i.e., n equals 5), or at least six cycles (i.e., n equals 6), and so on. As per the cutting method, a skilled person will appreciate that the number of cycles to be performed using the cookie cutter method may be varied based on the number of embryos to be produced from an initial donor embryo and the efficiency of embryo recovery following each cycle.

It is also envisaged that the number of portions into which each donor embryo is split using the “cookie cutter” method (i.e., the number of demi-embryos produced) may be varied within each cycle, as well as between cycles. In this regard, the number of portions into which a donor embryo is split may depend on the number of embryos to be produced and other factors, such as embryo health, embryo stage (e.g., embryonic cell numbers) etc.

Embryos that may be multiplied using the cut method and/or the cookie cutter method of the disclosure include embryos from the 2-cell stage through to blastocyst stage prior to implantation. In some examples, it may be advantageous to select a donor embryo for splitting which has a greater number of embryonic cells which are totipotent e.g., a blastocyst comprising between about 70-300 embryonic cells. In other examples, the donor embryo may be a late stage morula to early stage blastocyst e.g., comprising between about 30-70 embryonic cells. In yet another example, the donor embryo may be a morula e.g., comprising between about 16-32 embryonic cells. In yet another example, the donor embryo may be a pre-morula stage embryo comprising between 2 and about 16 embryonic cells. In each case, a skilled person will appreciate that the number of embryonic cells with the developing embryo at each stage may vary between species.

In each embodiment in which the one or more donor embryos are cut or split into multiple demi-embryos, the process of cutting (or splitting) the embryo may be performed using any means known in the art for splitting embryos. For example, the donor embryo may be split or cut by mechanical dissection using microsurgical instruments that rely on pressure, such as a blade (e.g., a scalpel blade or portion thereof) or a fine glass needle. Alternatively, or in addition, the donor embryo may be split or cut using a laser i.e., laser-assisted biopsy. In other examples, the donor embryo may be split or cut using a nano-dissection-based tool (e.g., using femtosecond laser pulse or atomic force microscopy (AFM) with a nano-scalpel). However, it is contemplated that any means known in the art may be employed.

In a further embodiment, the ‘twinning technique’ employed is designated “the unzip method”. In accordance with this embodiment, one or more of the embryonic cells are separated from the donor embryo by disrupting or “unzipping” the zona pellucida (ZP), and isolating one or more of the embryonic cells from within the donor embryo. In accordance with this embodiment, the zona pellucida of the donor embryo is disrupted to release the embryonic cells comprised therein, after which the embryonic cells are isolated (either singularly or in groups/cluster) and independently expanded in vitro to produce a plurality of embryos according to steps (i)-(iii) above. In this way, each isolated embryonic cell, or each cluster of embryonic cells (i.e., where two or more cells are isolated together) is expanded to become an embryo (e.g., a ZP-free embryo). The process is repeated in step (iv) using the newly produced embryos as the donor embryos. As indicated in step (v), the process can be repeated ‘n’ number of times (each referred to as a cycle), using the embryos produced from the previous cycle as donors embryos for the subsequent cycle. The number of cycles to be performed using the “unzip” method will depend on various factors including the number of embryos to be produced, the number of starting donor embryos, whether or not any embryos are harvested from the method during intervening cycles, and the number of embryonic cells in the donor embryos, the latter determining the upper limit of how many monozygotic embryos can be produced from any one donor embryo.

It is contemplated that the unzip method may be performed on any donor embryo comprising totipotent embryonic cells surrounded by a zona pellucida prior to complete compaction (i.e., 2-cell stage embryo to early pre-compacted morula stage embryo). In one example, the unzip method is performed using 2-cell donor embryos. In one example, the unzip method is performed using 4-cell donor embryos. In one example, the unzip method is performed using 8-cell donor embryos. In one example, the unzip method is performed using 16-cell donor embryos. In certain examples, the method is performed using donor embryos of varying developmental stages up to and including the early morula stage where compaction is not complete. In preferred examples, the embryonic cells within the zona pellucida are not compacted.

In one example, the one or more initial donor embryos each comprise at least 2 embryonic cells and the minimum number of cycles to be performed using the unzip method may be three i.e., n≥3.

In another example, the one or more initial donor embryos each comprise one or more embryonic cells that are developmentally equivalent to embryonic cells from a 16-cell embryo or a pre-compacted morula. In accordance with this example, the minimum number of cycles to be performed using the cutting method may be one i.e., n≥1. According to one example in which a single donor embryo comprising 16 blastomeres is used as the initial donor embryo, the unzip method may require a single cycle only in order to produce at least about 16 monozygotic embryos from the initial donor embryo. In another example, the unzip method may be performed starting with an initial donor embryo which comprises 8-12 blastomeres (i.e., an 8-cell stage embryo). In accordance with this example, the unzip method may require two cycles (i.e., n equals 2) in order to produce at least about 16 monozygotic embryos from the initial donor embryo. For example, two cycles of the unzip method in which the donor embryos at each cycle are “unzipped” at the 8-cell stage may be capable of producing about 64 to about 100 monozygotic embryos. Accordingly, it will be appreciated that fewer cycles may be necessary to achieve the desired number of embryos using the “unzip method” compared to the “cut method” and “cookie cutter method” as described herein.

As with the “cut method” and “cookie cutter method”, a skilled person will appreciate that the number of cycles to be performed using the “unzip method” may be varied based on the number of monozygotic embryos to be produced from an initial donor embryo and the number of embryonic cells in each donor embryo.

Disruption of the zona pellucida in the unzip method, also known as “assisted hatching”, may be performed using any suitable method known in the art. In this regard, a variety of techniques are known to be employed to assist embryo hatching in the field of assisted reproduction, including partial mechanical zona dissection, zona drilling and zona thinning, making use of acid tyrodes, proteinases, piezon vibrator manipulators and lasers e.g., as described in Hammadeh et al., (2011) J. Assist. Reprod. Genet., 28(2):119-128 It is also contemplated that the zona pellucida may be disrupted by nano-dissection (e.g., using femtosecond laser pulse or atomic force microscopy (AFM) with a nano-scalpel). Any one or more of the above-mentioned techniques may be employed in the unzip method of the disclosure for disrupting the zona pellucida.

Once the zona pellucida is disrupted, embryonic cells (e.g., blastomeres) may be isolated and, if appropriate, transferred to fresh culture media for expansion. A number of methods for isolating individual cells, including embryonic cells, are known in the art and contemplated herein (e.g., as described in Zhu and Murthy (2013) Curr. Opin. Chem. Eng., 2(1):3-7;). Techniques for isolating cells include, but are not limited to, fluorescence-activated cell sorting (FACS), magnetic-activated cell sorting (MACS), dielectrophoretic digital sorting, immunomagnetic cell separation, immunosurgery, hydrodynamic traps, laser capture microdissection, mechanical dissection, manual picking, microfluidics, micromanipulation, nanodissection, serial dilution, Raman tweezers and combinations thereof. Any one of these techniques, or a combination thereof, is contemplated for use in the methods of the disclosure to isolate single embryonic cells or aggregates of embryonic cells from the disrupted zona pellucida. In one particular example, microfluidics is employed to isolate individual embryonic cells.

In each of the embodiments of the method described herein, it may be desirable to immobilise the donor embryo(s) in order to cut or split the donor embryos or to enable disruption of the zona pellucida i.e., “unzip” the zona pellucida. Methods of immobilising embryos are known in the art and any one or more of those methods or techniques may be used in the method of the disclosure. Exemplary methods contemplated for use in the method of the disclosure include applying suction to the zona pellucida, making a depression or cul-de-sac in the container, constructing a device that traps the embryo, or making the embryo stick to a surface, e.g. by roughening the surface of the container containing the embryo, using protein-free culture medium or coating the culture container with a material which adheres to the outer membrane of the embryo.

As described herein, embryonic cells or demi-embryos comprising embryonic cells which have been separated from donor embryos using the method of the disclosure are cultured in vitro and expanded to produce a plurality of embryos e.g., monozygotic embryos.

Methodologies for culturing embryos in vitro at various stages of development are known in the art and contemplated herein. Exemplary methods are described in Examples 1-herein. A skilled person will appreciate that the culture conditions are important for growing the developing embryo to a blastocyst stage of development and may be varied/tailored according to the stage of embryonic development, as well as to control rate of embryonic development (e.g., cleavage) to provide sufficient windows of time to perform the multiplications steps of the method of the disclosure. For example, during embryo culture, variables such as temperature and CO₂ levels can be controlled to optimise growth of the developing embryo. For example, the optimum temperature for the development of an embryo is from about 32° C. to about 40° C., preferably from about 35° C. to 39° C., with a temperature of 37° C. being particularly preferred. The optimum CO₂ levels in the culturing environment for the development of an embryo is from about 1% CO₂ to about 10% CO₂, preferably from about 3% CO₂ to about 8% CO₂, and even more preferably about 5% CO₂.

Suitable media for culturing and expanding embryonic cells and embryos are known in the art. For example, culture media that allow embryos to mature to blastocysts at rates comparable with those that occur in vivo are described in Summers and Biggers (2003) Human Reprod Update, 9:557-582. Many of these culture media are based loosely on the concentrations of ions, amino acids, and sugars found in the reproductive tract of the female at the time of egg release, fertilization, and development (Gardner and Lane (1998) Hum Reprod 13:148-160). Typically, culture media containing a phosphate buffer or Hepes organic buffer are used for procedures that involve handling of gametes outside of the incubator, flushing of follicles and micromanipulation. Most culture media utilize a bicarbonate/CO₂ buffer system to keep PH in an suitable range e.g., pH 7.2-7.4. The osmolarity of the culture medium is typically in the range of 275-290 mosmol/kg. Embryos may also be cultured under paraffin oil (or alternative oil which is not toxic to embryos) to prevent evaporation of the medium preserving a constant osmolarity. The oil also minimizes fluctuations of pH and temperature when embryos are taken out of the incubator for microscopic assessment.

Suitable culture medium also typically contains a protein source, such as albumin or synthetic serum that is added at a concentration of about 5 to 20% (w/v or v/v, respectively). A source of salt may also added to the medium, such as NaCl, KCl, KH₂PO₄, CaCl₂2H₂O, MgSO₄7H2O, or NaHCO₃. Culture medium also typically contains a carbohydrates source, since carbohydrates are present in the female reproductive tract. Together with the amino acids they are the main energy source for the developing embryo. Culture media that support the development of zygotes up to 8-cells contain pyruvate and lactate. Some commercial media are glucose free, while others add a very low concentration of glucose to supply the needs of the sperm during conventional insemination. Media that support the development of 8-cell embryos up to the blastocyst stage contain pyruvate and lactate in low concentrations and a higher concentration of glucose. Supplement of the culture medium with amino acids may also be desirable for embryo development. Media that support the development of zygotes up to 8-cells are typically supplemented with non-essential amino acids such as proline, serine, alanine, aspargine, aspartate, glycine, and glutamate. Media that support the development of 8-cell embryos up to the blastocyst stage are also typically supplemented with essential amino acids, such as cystine, histadine, isolucine, leucine, lysine, methionine, valine, argentine, glutamine, phenylalanine, therionine, tryptophane. The culture medium may also contain vitamins.

The culture medium may also contain antibiotics. The majority of ART laboratories use indeed culture media containing antibiotics to minimize the risks of microbial growth. The most commonly used antibiotics being Penicillin ((3-lactam Gram-positive bacteria disturbs cell wall integrity) and Streptomycin (Aminoglycoside Gram-negative bacteria disturbs protein synthesis).

Three examples of sequential media for embryo development which may be useful in culturing embryos in the methods of the disclosure are: G1/G2 (Gardner et al, (1998) Hum. Reprod. 13:3434); Universal IVF Medium/MS (Bertheussen et al., (1997); and PI/Blastocyst Medium (Behr et al., (1998) Am. Soc. Rep. Med. 0-262). Media for culturing embryo at differing stages of development are commercially available from a range of sources.

Other exemplary culture media for embryo development are described in Examples 1-5 herein and are contemplated for use in the method of the disclosure.

In some example, the embryonic cells and/or developing embryos are cultured in the presence of one or more factors capable of promoting totipotency of the embryonic cells and/or inhibiting or preventing embryogenesis. Such factors may be added to culture media in order to prevent or slow down embryogenesis and thereby provide further opportunity to perform additional cycles of steps (i)-(iv) before cellular differentiation starts to occur. Factors which promote totipotency of embryonic cells and/or which inhibit or prevent embryogenesis are known in the art and contemplated for use herein. For example, factors that promote totipotency of embryonic cells and/or which inhibit or prevent embryogenesis include anti-Mirs and/or ribozymes that block miRNA stability or activity produced by the early embryo. Exemplary anti-Mirs may target miRNAs expressed by the embryo which promote clearance of maternal mRNAs (e.g., anti-Mirs that target the miR-30 family).

The skilled person will appreciate that the culture conditions may also contribute to maintaining the state of totipotency of the embryonic cells. Accordingly, during culture of the embryonic cells, embryos or demi-embryos, variables such as cell or embryo density, temperature and CO₂ levels can be controlled to moderate/control the rate of development of the cultured embryos.

As described herein, the method of the disclosure also comprises culturing and expanding the embryos in vitro to form blastocysts, which can then be harvested (e.g., for storage and/or implantation into a recipient female). Accordingly, at some stages of the method, the embryonic cells and/or developing embryos may be cultured in the presence of one or more factors capable of promoting embryogenesis. For example, factors capable of promoting embryogenesis (i.e., embryogenic factors) may be added to culture media used to culture embryos through to blastocyst stage for harvest. Factors which promote embryogenesis are known in the art and contemplated herein.

The skilled person will also appreciate that the culture conditions e.g., embryo density, temperature and CO₂ levels, can be varied and/or optimised in order to promote embryogenesis.

It is also contemplated that the various twinning techniques described herein i.e., the “cut method”, the “cookie cutter method” and the “unzip method”, may be used in combination with one another. For example, the method of the disclosure may comprise one or more cycles in which the donor embryos are split/cut into two or more demi-embryos using the “cut method” or “cookie cutter method”, followed by one or more cycles of the “unzip method” on the expanded demi-embryos produced by the early cycles. Combining these approaches may optimise the number of embryos produced whilst at the same time minimising the number of cycles that need to be performed. According to another example in which the techniques are combined, the method of the disclosure may comprise one or more cycles of the “unzip method” to produce a plurality of blastomeres (e.g., 16 blastomeres), followed by one or more cycles of the “cut method” or “cookie cutter method” on the embryos which are expanded from the blastomeres to thereby increase the number of embryos further by 2, 3 or 4 fold. In accordance with this last example, the one or more cycles of the “unzip method” may be performed in a laboratory and the subsequent cycle(s) of the “cut method” or “cookie cutter method” may be performed in the field prior to implantation. In this regard, the skilled person will appreciate that the various twinning techniques described herein may be combined in the method of the disclosure.

As described herein, the donor embryo obtained at step (i) of the method may be of any embryonic stage that comprises a plurality of totipotent embryonic cells e.g., 2-cell stage embryo to a blastocyst stage embryo prior to implantation. However, embryos of a particular developmental stage and/or comprising a particular number of embryonic cells may be preferred for certain embodiments of the method as described herein. To illustrate this point, embryos which are pre-compaction are preferred when performing the “unzip method” to enable isolation of the blastomeres. In some examples, the donor embryo is at a 2-cell stage. In some examples, the donor embryo is at a 4-cell stage. In some examples, the donor embryo is at an 8-cell stage. In some examples, the donor embryo is at an 16-cell stage. In some examples, the donor embryo is a morula. In other examples, the donor embryo is blastocyst (prior to hatching). Accordingly, a donor embryo useful in the method may comprise about 2 to 300 embryonic cells, such as e.g., about 4 to about 256 embryonic cells, or about 100 to about 256 embryonic cells, or about 70 to about 100 embryonic cells, or about 30 to about 70 embryonic cells, or about 16 to about 30 embryonic cells, or about 4 to about 16 embryonic cells, or about 4 to about 8 embryonic cells, or about 2 to about 4 embryonic cells.

As described herein, the species of animal from which the donor embryo(s) is/are obtained may be any vertebrate animal, including a species of mammal, a species of amphibian, a species of reptile, a species of fish and a species of bird (e.g., poultry).

In one example, the animal is a mammal e.g., a non-human mammal. Exemplary non-human mammals for which the method of the disclosure may be useful include livestock species (e.g., cattle, buffalo, pigs, sheep, goats, camelid, deer, horses etc.), companion animals (e.g., dogs, cats etc.), laboratory animals (e.g., rats, mice, hamsters, guinea pigs, rabbits, etc.), non-human primates (macaque and marmoset etc.) and wildlife species (e.g., marsupials, cats, rhino, giant panda etc.).

In one particular example, the method of the disclosure may be used to produce a plurality of embryos (e.g., monozygotic embryos) in a bovine species. In another example, the method of the disclosure may be used to produce a plurality of embryos (e.g., monozygotic embryos) from sheep. In another example, the method of the disclosure may be used to produce a plurality of embryos (e.g., monozygotic embryos) from pigs. In another example, the method of the disclosure may be used to produce a plurality of embryos (e.g., monozygotic embryos) from goats. In another example, the method of the disclosure may be used to produce a plurality of embryos (e.g., monozygotic embryos) from horses. In another example, the method of the disclosure may be used to produce a plurality of embryos (e.g., monozygotic embryos) in a camelid species.

Donor embryo used in the first cycle of the method of the disclosure may be prepared in vivo (e.g., by conventionally flushing embryos from a pregnant animal) or by in vitro fertilisation (IVF) methods.

In one example, the donor embryo used in the first cycle of the method is prepared by in vivo methods. For example, an oocyte may be fertilized in vivo (e.g., following copulation or by artificial insemination) and subsequent embryos retrieved from the pregnant female by conventional embryo flushing. In one example, the donor embryos are produced by multiple ovulation embryo transfer (MOET), whereby the donor female is administered hormones prior fertilisation, primarily follicle stimulating hormone (FSH), to stimulate the ovaries of the cycling female animal to induce multiple ovulations.

In another example, the donor embryo used in the first cycle of the method is prepared by in vitro methodologies i.e., IVF. Methods for producing embryos using IVF are well known in the art. IVF generally involves the production of oocytes from donor animals by follicle aspiration, following by in vitro maturation, fertilisation and culture until the resulting embryos have reached a desired developmental stage. Conveniently, this approach permits the repeated production of embryos from live animals of particular value under controlled conditions. Methods for IVF production of embryos are described in Berlinguer F. “Embryo Production”, In: Animals Production in Livestock, Encyclopedia of Life Support Systems (EOLSS), the full content of which is incorporated herein.

It is also contemplated that the donor embryo used in the first cycle of the method, whether produced by in vivo or in vitro means, may be fresh or thawed (i.e., a thawed cryopreserved embryo). In one example, the donor embryo is fresh. In another example, the donor embryo is a thawed cryopreserved embryo.

Donor embryos useful in the method of the disclosure may also have undergone genetic modifications. For example, embryonic cells within the donor embryo may be genetically modified prior to performance of the method such that all embryos produced from the donor carry the genetic modification. In one example, the donor embryo is genetically modified by introducing an exogenous nucleic acid to the genome of the embryonic cells comprised therein. The exogenous nucleic acid may be an alternative allele for a gene or loci associated with a trait of interest. Alternatively, the exogenous nucleic acid may be a transgene. In another example, the donor embryo may be genetically modified by editing the genome of the embryonic cells comprised therein (i.e., genome editing). The genome edit may be selected from the group consisting of an insertion, deletion, substitution, inversion or translocation. For example, the genome edit may be an insertion, deletion and/or substitution of a nucleic acid sequence, or one or more nucleotide positions therein, in order to replace an existing allele of a gene or loci associated with a trait of interest with an alternative allele.

Genome editing may also be employed to introduce one or more genetic modifications (e.g., nucleotide substitutions) which, considered alone or in combination, provide a unique genetic profile or fingerprint in the developing embryo. This unique genetic profile or fingerprint can then be used to identify and/or trace embryos produced from the donor embryo (and animals produced therefrom). For example, one or more conservative nucleotide substitutions within safe harbour regions of genome may be made to embryonic cells within the donor embryo in order to generate unique genetic profiles or fingerprints.

Preferably, the genetic modification or editing occurs at the single cell stage such that all subsequent cells in the developing embryo derived from the modified cell (and animal resulting therefrom) comprise the modification. If, however, the genetic modification event occurs after one or more cell divisions, and not all embryonic cells within the donor embryo are modified, then the donor embryo may be a mosaic for the modification/edit event, in that it will have some cells derived from the modified/edited cell and some cells derived from unmodified/unedited cells.

A number of methods for genetically modifying genomes of a cell using targeted nucleases are described in the art. These include but are not limited to (1) clustered regularly interspaced short palindromic repeats (CRISPR)-CRISPR-associated protein 9 (Cas9) or other Cas systems, (2) transcription activator-like effector nucleases (TALENs), (3) zinc-finger nucleases (ZFNs), and (4) homing endonucleases or meganucleases. These are other methods for genetically modifying cells are contemplated for use in the method of the disclosure in order to genetically modify donor embryos.

The methods of the disclosure may further comprise one or more steps to assist with selection of donor embryos to be multiplied using the method. For example, the method may comprise selecting the donor embryo prior to step (i) on the basis of one or more genetic screening criteria, genetic diagnoses and/or one or more morphological criteria.

In one example, genetic screening criteria may be determined by screening for the presence or absence of one or more genetic markers (e.g., SNP alleles or haplotype) associated with (favourable variant of) a phenotypic trait of interest e.g., a commercially-important production trait as may be the case for a livestock species. Exemplary phenotypic traits of interest include, but are not limited to, production traits (e.g., growth rate, fecundity, feed conversion efficiency etc.), drug resistance, susceptibility to pests and/or parasites, and sex (i.e., male or female). In this way, donor embryos for multiplication using the method of the disclosure can be obtained from elite animals.

Alternatively, or in addition, the donor embryo may be selected on the basis of a genetic diagnosis for one or more conditions, diseases or predisposition thereto. In this regard, preimplantation genetic diagnosis (PGD) of embryos has become more common place in the field of IVF. PGD tests have largely focused on two methodologies: fluorescent in situ hybridization (FISH) and polymerase chain reaction (PCR). However, a number of techniques for PGD are known in the art and one or more of these techniques may be used in the method of the disclosure to select donor embryos. These include, but are not limited to, methods which rely on polymerase PCR, FISH, single strand conformational polymorphism (SSCP), restriction fragment length polymorphism (RFLP), primed in situ labelling (PRINS), comparative genomic hybridisation (CGH), COMET analysis (single cell gel electrophoresis), heteroduplex analysis, Southern analysis, and denaturing gradient gel electrophoresis (DGGE) analysis.

Alternatively, or in addition, the donor embryo may be selected on the basis of one or more morphological characteristics, such as morphological characteristics which are indicative of embryo health.

As described herein, once a desired number of embryos (e.g., monozygotic embryos) are produced using the method of the disclosure, those embryos may be matured to a desired embryonic developmental stage in vitro (e.g., preimplantation blastocyst) and harvested from the culture media. Harvested embryos may then be stored in an appropriate embryo holding or transfer media until they are transferred to recipient females and/or until such a time as the embryo is cryopreserved. Any commercially available embryo holding and transfer media is contemplated for use herein. In accordance with examples in which the embryos are to be placed in short term storage prior to transfer to a recipient female (such as during transport), the harvested embryos may be stored at between about 2° C. to about 8° C. depending on the specifications of the particular holding or transfer media. In some preferred examples, the harvested embryos are stored at about 4° C.

Harvested embryos may also be cryopreserved for storage. The main techniques used in the art for embryo cryopreservation are vitrification and slow programmable freezing (SPF), both of which are contemplated herein. In accordance with this example, the harvested embryos can be transferred to an appropriate cryopreservation media (e.g., containing ethylene glycol freeze media or similar), cryopreserved, and maintained at about −180° C. to about −196° C. until they are thawed for use and/or they are shipped. For example, the cryopreserved embryos may be stored in liquid nitrogen at about −196° C.

In addition to the application of the method of the disclosure in commercial livestock breeding, it is also contemplated that the method of the disclosure may have applications in the area of animal conservation and management. For example, embryos produced from donor embryos obtained from endangered or threatened species (including wildlife and domesticated species) using the method of the disclosure may be deposited with biobanks and/or disseminated for breeding programs. This may assist with breeding programs and management of populations of endangered or threatened species. Accordingly, in some examples, the method may further comprise depositing one or more cryopreserved embryos prepared by the method of the disclosure with a biobank.

In accordance with embodiments in which the harvested embryos are transferred fresh to recipient females, the method of the disclosure may further comprise transferring one or more of the monozygotic embryos to the oviduct(s) of one of more recipient females. Methods for embryo transfer are known in the art. For example, embryos may be manually transferred using a catheter or other means.

Accordingly, the present disclosure also provides a method of breeding an animal, comprising:
(i) transferring one or more of the plurality of embryos produced by the method described herein to the oviduct(s) of one of more recipient females to establish a pregnancy; and
(ii) producing the animal from the pregnant recipient female by parturition.

As described herein, the animal may be any vertebrate animal, including a species of mammal, a species of amphibian, a species of reptile, a species of fish and a species of bird (e.g., poultry). In one particular example, the animal is a mammal e.g., anon-human mammal. Exemplary non-human mammals for which the method of the disclosure may be useful include livestock species (e.g., cattle, buffalo, pigs, sheep, goats, camelid, deer, horses etc.), companion animals (e.g., dogs, cats etc.), laboratory animals (e.g., rats, mice, hamsters, guinea pigs, rabbits, etc.), non-human primates (macaque and marmoset etc.) and wildlife species (e.g., marsupials, cats, rhino, giant panda etc.). In one particular example, the method of the disclosure may be used to breed cattle. In another example, the method of the disclosure may be used to breed sheep. In another example, the method of the disclosure may be used to breed pigs. In another example, the method of the disclosure may be used to breed goats. In another example, the method of the disclosure may be used to breed horses. In another example, the method of the disclosure may be used to breed camelids (e.g., alpacas).

### EXAMPLES

### Example 1: Serial Twinning of Embryos Using the “Cut Method”

This example outlines the experimental steps for performing the “cut method” to produce multiple embryos e.g., monozygotic embryos.

36 donor (blastocyst) embryos obtained from Ultrablack cows produced by MOET are obtained from Nindooinbah (Beaudesert, QLD, Australia). The 36 MOET embryos are divided into the following test groups:
1. Control group—six embryos to remain uncut
2. Test group 1—six embryos to undergo a single bisection
3. Test group 2—six embryos to undergo two serial bisections (i.e., two consecutive cycles of bisection and expansion)
4. Test group 3—six embryos to undergo three serial bisections (i.e., three consecutive cycles of bisection and expansion)
5. Test group 4—six embryos to undergo four serial bisections (i.e., four consecutive cycles of bisection and expansion)
6. Test group 5—six embryos to undergo five serial bisections (i.e., five c consecutive cycles of bisection and expansion)

For test group 1, donor blastocyst embryos are bisected by microdissection. Following bisection, embryo halves are cultured for 2, 4 or 6 days to assess restoration of inner cell mass (ICM) numbers, trophoblast numbers and overall embryo survival. Analysis is performed on fixed embryos via morphological embryo scoring, immunohistochemistry for ICM markers (e.g., Nanog, SOX2, OCT4 etc), trophoblast markers (CDX2) and live/dead cell staining to determine optimal culture conditions. Results are validated by qPCR on RNA isolated from the respective 3-8 embryos.

Culture conditions identified in test group 1 as being optimal for expansion of the ICM in bisected embryos are taken forward in test groups 2-5. Success of serial bisection is assessed using the analysis described for test group 1 above.

For each of test groups 3, 4 and 5, embryos which have expanded and appear to be healthy following the serial bisection process are implanted into recipients to assess pregnancy rates and healthy development of offspring.

### Example 2: Production of Multiple Monozygotic Embryos Using the “Cookie Cutter Method”

This example outlines the experimental steps to be undertaken when performing the “cookie cutter method” for production of multiple monozygotic embryos.

24 donor (blastocyst) embryos obtained from Ultrablack cows produced by MOET are obtained from Nindooinbah (Beaudesert, QLD, Australia). The 24 MOET embryos are divided into the following test groups:
1. Control group—six embryos to remain uncut
2. Test group 1—six embryos to undergo bisection
3. Test group 2—six embryos to be quartered
4. Test group 3—six embryos to be cut into eighths

All embryo splitting is performed by a Veterinarian at Nindooinbah according to the test group criteria.

The six embryos in the control group are uncultured, whilst the embryo pieces/sections produced by splitting are cultured for up to seven days to assess restoration of inner cell mass numbers, trophoblast numbers and embryo survival.

Following culture, analysis is performed on 3-8 fixed embryos via morphological embryo scoring, immunohistochemistry for ICM markers (e.g., Nanog, SOX2, OCT4 etc), trophoblast markers (CDX2) and live/dead cell staining. Results are validated by qPCR on RNA isolated from the respective 3-8 embryos.

### Example 3: Production of Multiple Monozygotic Embryos Using the “Unzip Method”

This example outlines the experimental steps for performing the “unzip method” to produce multiple monozygotic embryos from a single donor embryo.

A mobile laboratory at Nindooinbah (Beaudesert, QLD, Australia) is used to supply timed fertilized oocytes inseminated with semen from a single bull: eight 4-cell donor and four 8-cell embryos are provided.

Pronase treatment of the embryos according to a published protocol is used to disrupt the zona pellucida of the embryos, after which time single blastomeres and dual blastomere aggregates are isolated and deposited in separate wells of a multi-well plate containing culture media for expansion.

Following culture, analysis is performed on fixed embryos via morphological embryo scoring, immunohistochemistry for ICM markers (e.g., Nanog, SOX2, OCT4 etc), trophoblast markers (CDX2) and live/dead cell staining. Results are validated by qPCR on RNA isolated from the respective 3-8 embryos.

The 12 best looking expanded blastomere aggregates are cultured up to 7 days, and the best 4 looking blastocysts (whether derived from a single blastomere or duel blastomeres) are implanted into recipients to assess pregnancy rates and healthy development of offspring.

### Example 4: Production of Multiple Embryos Through Serial Splitting

This example describes experiments in which the inventors performed serial multiplication of donor embryos (also referred to herein as conceptuses) using the “unzip method” to produce multiple blastocysts. This example demonstrates that the serial multiplication method of the disclosure, in this case using the “unzip” technique, is able to significantly improve the efficiency of blastocyst production relative to standard culturing of intact conceptuses without multiplication.

### Methods

### Conceptus Culture Media Preparation

### Chemicals and Stock Solutions

Stock solutions for culture and unzipping media were prepared according to Table 1 unless described otherwise; all media reagents used in this study were obtained from Sigma.

Stock solutions were prepared using water. The vapour pressure osmometer (Wescor) was used to adjust the osmolality of NaCl, KCl and NaHCO₃ to 2000 mOsm, 200 mOsm and 2000 mOsm respectively, using MilliQ® water.

TABLE 1
Stock solutions used to prepare media for culture and unzipping.

| | Final | Amount/volume | |
|---|---|---|---|
| Compound | concentration | of H₂O | Storage |
| NaCl | 1M | 2920 mg/50 mL | Stored at 4° C. |
| KCl | 0.1M | 370 mg/50 mL | Stored at 4° C. |
| Na₂EDTA | 0.001M | 19 mg/50 mL | Stored at 4° C. |
| HEPES | 0.154M | 1835 mg/50 mL | Stored at 4° C. |
| Myo-inostiol | 0.277M | 998 mg/20 mL | Aliquoted and |
| | | | stored at −20° C. |
| Na₃citrate•2H₂O | 0.05M | 294 mg/20 mL | Aliquoted and |
| | | | stored at −20° C. |
| KH₂PO₄ | 0.1M | 136 mg/10 mL | Stored at 4° C. |
| MgSO₄ | 0.1M | 246 mg/10 mL | Stored at 4° C. |
| CaCl₂•2H₂O | 0.17M | 251 mg/10 mL | Stored at 4° C. |
| NaOH | 2M | 800 mg/10 mL | Stored at 4° C. |
| HCl | 1M | 1 mL/10 mL | Stored at 4° C. |
| NaHCO₃ | 1M | 420 mg/5 mL | Freshly made for |
| | | | one week use |
| Na Pyruvate | 0.1M | 55 mg/5 mL | Freshly made for |
| | | | one week use |
| Glucose | 0.1M | 90 mg/5 mL | Freshly made for |
| | | | one week use |
| Penicillin/ | 6 mg/mL | 30 mg/5 mL | Freshly made for |
| Gentamicin | 5 mg/mL | 25 mg/5 mL | one week use |
| L-alany1-L- | 0.5M | 543 mg/5 mL | Aliquoted and |
| glutamine | | | stored at −20° C. |

### Preparation of Media for Conceptus Culture and Unzipping

NbryoIVC-2 Ca²⁺ medium was used for conceptus unzipping procedures and NbryoIVC-3 medium was used for conceptus culture before and after unzipping (until Day 8 of development). Media were prepared by adding stock solutions according to the listed order in Table 2. The pH of media were adjusted to 7.4 by adding 2 M NaOH. Media were tested for osmolality using an osmometer (Wescor). Osmolality was adjusted to 270 mOsm by the addition of MilliQ® water. Lastly, fatty acid free bovine serum albumin (FAF-BSA) was added to media at a concentration of 4 mg/mL and media were filter-sterilized with a 0.22 μm syringe filter (Millipore). Media were stored at 4° C. for a maximum of two weeks.

TABLE 2
Composition of NbryoIVC-2 Ca²⁺ free and NbryoIVC-3 media

| | |
|---|---|
| NbryoIVC-2 Ca²⁺ free | NbryoIVC-3 |

| Component | Final concentration | | | |
|---|---|---|---|---|
| Lactic Acid (60%) | 4.92 | mM | 4.92 | mM |
| NaCl | 100 | mM | 100 | mM |
| KCl | 6.64 | mM | 6.64 | mM |
| KH₂PO₄ | 1.10 | mM | 1.10 | mM |
| MgCl₂•6H₂O | 0.45 | mM | 0.45 | mM |
| NaHCO₃ | 23.27 | mM | 23.27 | mM |
| Na₂EDTA | 0.1 | mM | — | |
| Na Pyruvate | 0.4 | mM | 0.4 | mM |

| | | |
|---|---|---|
| Penicillin or | 0.06 mg/mL or | 0.06 mg/mL or |
| Gentamicin | 0.05 mg/mL | 0.05 mg/mL |

| | | | | |
|---|---|---|---|---|
| Myo-inositol | 2.56 | mM | 2.56 | mM |
| Na₃-citrate | 0.46 | mM | 0.46 | mM |
| H₂O | 20 | mL | 20 | mL |

| CaCl₂•2H₂O | — | 1.09 | mM |
|---|---|---|---|
| MEM Essential amino | — | 0.93X | |
| acids | | | |
| MEM non-essential | — | 0.93X | |
| amino acids | | | |
| L-alany1-L-glutamine | — | 1 | mM |

### Pronase Preparation

Pronase is a proteolytic enzyme used for the removal of the zona pellucida (ZP) during unzipping procedures. Pronase was prepared at a final concentration of 0.3 mg/mL in HEPES buffered-NbryoIVC-3. It was then filter-sterilized through a 0.22 μm syringe filter, aliquoted, and stored at −20° C.

### In Vitro Development of Bovine Conceptuses

### Production of Bovine Zygotes

Bovine zygotes were produced by IVF using commercial protocols of ArtSolutions. Bovine oocytes were matured in vitro (IVM) from ovaries collected from Nindoonibah Cattle Farm following standard procedures. After 24 hours of IVM, matured oocytes were then in vitro fertilized with thawed semen from a single bull of proven fertility from Nindoonibah Cattle Farm. Following 24 hours of IVF, the presumptive zygotes were moved to VitroCleave (ArtSolutions) in vitro culture (IVC) medium. Unless stated otherwise, zygotes were cultured in VitroCleave (ArtSolutions) IVC medium at 38.5° C. under 7% 02 and 5% CO₂. Zygotes were cultured to the 2-cell, 4-cell, 8-cell, or 16-cell for 25-32 h, 32-42 h, 42-52 h, 96-100 h, respectively, after IVF.

### Preparation of Plates and Dishes for Unzipping

Pre-Coating Plates with 0.1% PVA

To avoid adherence of ZP-free conceptuses to the plate, wells of 96-well round bottom plates (Corning) were coated by adding 50 μL of sterile 0.1% PVA in sterile water to each well and allowed to incubate overnight at 38.5° C. Each well was then washed three times with sterile water to remove unbound PVA. The plates were then dried, sealed and stored at 4° C. until use.

### Unzipping Dishes

Prior to the unzipping procedure, a 55 mm Petri dish (Corning) containing 20 μL droplets of pronase, NbryoIVC-2 Ca²⁺ free, and NbryoIVC-3 media overlaid with mineral oil (Coopers Scientific), was equilibrated at 38.5° C. under 7% 02, 5% CO₂ for at least 60 min.

### Post-Unzipping Culture System

The 96-well plates pre-coated with 0.1% PVA were utilized for the culture of individual blastomere after unzipping. Each well contained 50 μL of NbryoIVC-3 or 20 μL VitroBlast (ArtSolutions) IVC media, and overlaid with mineral oil to avoid medium evaporation. Plates with media were equilibrated for at least 60 minutes at 38.5° C. under 7% O₂, 5% CO₂ prior to transferring separated blastomeres into each well.

### Serial Unzipping Procedure

All bovine conceptus-unzipping procedures were performed under a dissecting microscope with a plate heated to 37° C. In the first serial unzipping (serial n=1), either 2-, 4-, 8- or 16-cell conceptuses were treated with pronase, to remove the surrounding ZP, for 2 minutes at 38.5° C. in a humidified incubator in an atmosphere of 7% O₂ and 5% CO₂. Once the ZP dissolved, conceptuses were washed through three 20 μL drops of NbryoIVC-3 medium to rinse off any remaining pronase and incubated for 10 minutes at 38.5° C. under 7% 02 and 5% CO₂. ZP-free conceptuses were then transferred to NbryoIVC-2 Ca²⁺ free medium for 3 minutes at 38.5° C. under 7% 02 and 5% CO₂ to decrease cell-cell contact. Then, in the NbryoIVC-2 Ca′ free medium blastomeres in each conceptus were separated by aspiration using a micropipette (˜120 μm diameter). Individual blastomeres were individually cultured in PVA pre-coated wells containing allocated medium under 7% O₂, 5% CO₂, at 38.5° C.

Blastomeres going through several rounds of unzipping procedures (serial n=2, serial n=3 or serial n=4) were unzipped subsequent to division. In the second unzipping procedure (serial n=2), cleaved blastomeres were placed in NbryoIVC-2 Ca′ free medium for 1-3 minutes at 38.5° C. under 7% O₂ and 5% CO₂. As previously mentioned, by aspiration using a micropipette, blastomeres were separated and individually cultured in PVA pre-coated wells containing allocated medium. This process was repeated in the third unzipping procedure (serial n=3) and the fourth unzipping procedure (serial n=4). Blastomeres were scored every 12-24 hours until Day 8 of preimplantation development according to their developmental status (cleaved, compacted, cavitated and small blastocyst). Conceptuses were classified as a blastocyst when the cavity is greater than half the volume of the conceptuses and there is a cohesive cluster of ICM cells.

### Nomenclature and Definitions for Example 4

As described herein, the term “embryo” refers to the zygote that is formed when two haploid gametic cells (e.g., an unfertilized oocyte and a sperm cell) unite to form a diploid totipotent cell (e.g., a fertilized ovum), as well as to the embryo that results from the subsequent cell divisions (i.e. embryonic cleavage), including the morula stage (i.e. about 16-cell stage) and blastocyst stage with differentiated trophoectoderm and inner cell mass. A “conceptus” as described herein is the developing embryo from fertilization until the appearance of the primitive streak (equivalent of Day 18 of development in bovine). Since the inventors cultured bovine zygotes and individual blastomeres up to eight days post-fertilization (to the blastocyst stage), the term “conceptus” has been used to describe the developing entity.

Blastomeres isolated from the 2-, 4-, 8-, 16-cell stage conceptuses are referred to herein as “1:2”, “1:4”, “1:8”, and “1:16” blastomeres, respectively, wherein the numerator denotes the number of blastomeres in the cell mass and the denominator denotes the equivalent conceptus stage the blastomeres. FIG. 1 illustrates this nomenclature system with regards to the unzipping of 2-cell conceptuses via serial n=4 unzipping rounds. FIG. 1 is schematic representation of the classification scheme for blastomeres and developing embryos during 4 rounds of unzipping from the 2-cell stage embryos. The left hand side of FIG. 1 represents normal development of preimplantation conceptus development from the zygote stage to the blastocyst stage. During the unzipping procedure, which is shown on the right hand side of FIG. 1, the ZP is removed to separate individual blastomeres within the conceptus (Serial n=1). Individual blastomeres isolated from the 2-cell conceptus are referred to as 1:2. These blastomeres are then allowed to develop to form pairs, named 2:4. Subsequent to cleavage, these 2:4 blastomeres can be taken to another round of unzipping (Serial N=2), where the blastomeres will be separated to 1:4. This process can be repeated n times e.g., n=3 and/or n=4 or more. As these blastomeres develop, they transition to the subsequent equivalent preimplantation conceptus stage and therefore the denominator changes accordingly. After the 1:16 stage, blastomeres can compact, cavitate to then form a blastocyst equivalent.

### Results

Unzipping 2-Cell Bovine Conceptuses Via Serial n=4 Unzipping Procedures.

A total of 28 2-cell bovine conceptuses were subjected to unzipping. After the serial n=1 unzipping procedure, 56 1:2 blastomeres were obtained (See Table 2; and FIG. 1 for explanation of nomenclature). 55/56 of the 1:2 blastomeres divided into 2:4, of which 48 were taken to another unzipping round, serial n=2. After serial n=2 unzipping, 91/96 of the 1:4 blastomeres divided into 2:8, of which 81 were taken to another unzipping round, serial n=3. After serial n=3 unzipping procedure 145/162 of the 1:8 blastomeres divided into 2:16, of which 100 were taken to another unzipping round, serial n=4. All of the individual blastomeres in serial n=1, n=2, and n=3 were cultured in 50 μL of NbryoIVC-3 medium. Lastly, after serial n=4 unzipping procedure, 199 1:16 blastomeres were obtained and were left to progress to the blastocyst equivalent stage in 20 μL of VitroBlast (ArtSolutions) IVC medium. Out of these 199 1:16 blastomeres, 171 (85.9%) divided, 156 (78.4%) compacted, 127 (63.8%) cavitated and 53 (26.6%) progressed to form a small blastocyst.

The literature consistently reports that the intact bovine conceptuses are cultured to the blastocyst stage with efficiency of ˜30% (as shown in Table 2), and this is the experience of the inventors who have extensive experience in embryo culture and transfer in livestock. Therefore, had the 28 2-cell donor conceptuses been cultured without performing the unzipping procedure described herein, a yield of 8 blastocysts would be expected on the basis of the ˜30% efficiency. Using the above serial unzipping procedure, the inventors were able to produce 53 blastocysts from the initial 28 donor conceptuses, thereby improving the efficiency of blastocyst production by approximately 6-fold relative to culturing of intact conceptuses.

TABLE 2
Development of blastocyst equivalents derived from unzipped 2-cell
bovine conceptuses via a serial unzipping procedure (serial n = 4)

| Number | | | |
|---|---|---|---|
| of serial | Entities at | Unzipped | Expected outcome for |
| unzips | various steps | conceptuses | intact conceptuses |
| n = 1 | No. of 2-cell | 28 | 28 |
| | conceptuses | | |
| | Blastomeres | 56 | |
| | obtained (1:2) | | |
| | Blastomeres | 55 | |
| | divided (2:4) | | |
| n = 2 | No. of pairs of | 48 | |
| | blastomeres unzipped | | |
| | Blastomeres | 96 | |
| | obtained (1:4) | | |
| | Blastomeres | 91 | |
| | divided (2:8) | | |
| n = 3 | No. of pairs of | 81 | |
| | blastomeres unzipped | | |
| | Blastomeres | 162 | |
| | obtained (1:8) | | |
| | Blastomeres | 145 | |
| | divided (2:16) | | |
| n = 4 | No. of pairs of | 100 | |
| | blastomeres unzipped | | |
| | Blastomeres | 199 | |
| | obtained (1:16) | | |
| | Blastomeres | 171/199 | |
| | divided (%) | (85.9%) | |
| | No. of | 156/199 | |
| | compacted (%) | (78.4%) | |
| | No. of | 127/199 | |
| | cavitated (%) | (63.8%) | |
| | No. of blastocyst | 53/199 | 8/28 |
| | equivalent (%) | (26.6%) | (~30.0%) |

### Example 5: Production of Multiple Embryos Via Unzipping of ≥8-Cell Bovine Embryos

This example describes experiments in which the inventors performed unzipping of ≥8-cell donor conceptuses using the “unzip method” to produce multiple blastocysts.

### Methods

Culture media, culture conditions and the procedure for unzipping conceptuses were generally as described in the ‘methods’ section of Example 4.

Briefly, a total of 19 bovine≥8-cell stage conceptuses were subjected to unzipping. Conceptuses unzipped contained between 8 and 43 cells. Separated blastomeres were individually cultured in 20 μL, of VitroBlast (ArtSolutions) IVC medium. Since cleavage of cells in the conceptus is not synchronous, heterogenous stages of blastomere development exist in these conceptuses, comprising of 1:8, 1:16, 1:32 and 1:64 blastomeres. As a result, each type of blastomere was analysed separately.

### Results

From 19 conceptuses, 53 1:8 blastomeres were obtained, of which 38 (71.7%) divided, 34 (64.1%) compacted, 25 (47.2%) cavitated and 21 (39.6%) formed a blastocyst (Table 2.2). In addition, 162 1:16 blastomeres were obtained, of which 126 (50%) divided, 105 (64.8%) compacted, 99 (61.1%) cavitated and 36 (22.2%) formed a blastocyst. 54 1:32 blastomeres were obtained, of which 47 (87.0%) divided, 45 (83.3%) compacted, 44 (81.4%) cavitated and 3 (5.6%) formed a blastocyst. Some of the 1:32 blastomeres were cultured in pairs (i.e., 2:32). Specifically, two 2:32 blastomere pairs were obtained from the unzipping procedure, of which 2 (100%) divided, 1 (50.0%) compacted, 1 (50.0%) cavitated and none formed a blastocyst. Lastly, 27 1:64 blastomeres were obtained, of which 13 (48.1%) divided, 11 (40.7%) compacted, 11 (40.7%) cavitated and 1 (3.7%) formed a blastocyst. Altogether, from the original 19 donor conceptuses, 61 blastocysts were yielded. This is summarised in Table 3.

Starting with ≥8-cell stage conceptuses, including conceptuses comprising blastomeres which are developmentally equivalent to 16-, 32- and 64-cell embryos, the inventors have shown embryo multiplication using the “unzip” method is capable of significantly increasing efficiency of blastocyst production. In this regard, the inventors were able to produce 61 blastocysts from the initial 19 donor conceptuses, which represents a 10-fold increase in efficiency of blastocyst production relative to culturing of intact conceptuses (which typically achieves an efficiency of ˜30%, discussed above).

TABLE 3
Development of blastocyst equivalents derived from
unzipped ≥8-cell bovine conceptuses via one serial
unzipping procedure (serial N = 1)

| | | 1:8 | 1:16 | 1:32 | 2:32 | 1:64 |
|---|---|---|---|---|---|---|
| Serial | No. of | 19 | | | | |
| N = 1 | conceptuses | | | | | |
| | Blastomeres | 53 | 162 | 54 | 2 | 27 |
| | obtained | | | | | |
| | Blastomeres | 38/53 | 126/162 | 47/54 | 2/2 | 13/27 |
| | divided (%) | (71.7%) | (77.8.0%) | (87.0%) | (100.0%) | (48.1%) |
| | No. of | 34/53 | 105/162 | 45/54 | 1/2 | 11/27 |
| | compacted | (64.1%) | (64.8.0%) | (83.3%) | (50.0%) | (40.7%) |
| | (%) | | | | | |
| | No. of | 25/53 | 99/162 | 44/54 | 1/2 | 11/27 |
| | cavitated | (47.2%) | (61.1%) | (81.4%) | (50.0%) | (40.7%) |
| | (%) | | | | | |
| | No. of | 21/53 | 36/162 | 3/54 | 0/2 | 1/27 |
| | blastocyst | (39.6%) | (22.2%) | (5.6%) | (0.0%) | (3.7%) |
| | equivalent | | | | | |
| | (%) | | | | | |

It will be appreciated by persons skilled in the art that numerous variations and/or modifications may be made to the above-described embodiments, without departing from the broad general scope of the present disclosure. The present embodiments are, therefore, to be considered in all respects as illustrative and not restrictive.

1. A method of multiplying one or more donor embryos, said method comprising:
(i) obtaining one or more donor embryos comprising at least two embryonic cells;
(ii) separating one or more of the embryonic cells from the one or more donor embryos;
(iii) expanding the embryonic cells in vitro under conditions suitable to produce a plurality of embryos, each comprising at least two embryonic cells;
(iv) isolating one or more of the plurality of embryos produced at (iii) to be used as donor embryos in subsequent multiplications; and
(v) repeating steps (i)-(iv) ‘n’ times, wherein ‘n’ is ≥3.

2. The method of claim 1, wherein:
n is equal to ≥4;
16 or more monozygotic embryos are produced from a donor embryo obtained at (i); and/or
the one or more donor embryos each comprise 2 to 64 embryonic cells.

3. (canceled)

4. (canceled)

5. The method of claim 1, wherein the one or more donor embryos each comprise 2 to 16 embryonic cells.

6. A method of multiplying a donor embryo, said method comprising:
(i) obtaining a donor embryo comprising one or more embryonic cells that are developmentally equivalent to embryonic cells from a 16-cell embryo or a pre-compacted morula;
(ii) separating one or more of the embryonic cells from the donor embryo;
(iii) expanding the embryonic cells in vitro under conditions suitable to produce a plurality of monozygotic embryos from the donor embryo; and
(iv) culturing the plurality of monozygotic embryos under conditions suitable to produce a plurality of monozygotic blastocysts.

7. The method of claim 6, wherein prior to the step of culturing the plurality of monozygotic embryos to produce the plurality of blastocysts, the method further comprises the steps of:
(i) isolating one or more of the plurality of monozygotic embryos produced to be used as donor embryos in subsequent multiplications, wherein each donor embryo isolated for subsequent multiplications comprises at least two embryonic cells;
(ii) separating one or more of the embryonic cells from the one or more donor embryos;
(iii) expanding the embryonic cells in vitro under conditions suitable to produce a plurality of embryos, each comprising at least two embryonic cells;
(iv) isolating one or more of the plurality of embryos produced at (iii) to be used as donor embryos in subsequent multiplications; and
(v) repeating steps (i)-(iv) ‘n’ times before culturing the plurality of embryos under conditions suitable to produce a plurality of blastocysts.

8. The method of claim 7, wherein n is equal to ≥2, or n is equal to ≥3, or n is equal to ≥4.

9. (canceled)

10. (canceled)

11. The method of claim 1, wherein separation of the one or more embryonic cells from the one or more donor embryos is achieved by:
(i) splitting the donor embryo into a plurality of portions, each portion comprising one or more embryonic cells; or
(ii) disrupting a zona pellucida (ZP), and isolating the one or more of the embryonic cells from the one or more donor embryos.

12. The method of claim 11, wherein:
(i) splitting the one or more donor embryos is performed using a microsurgical blade, or laser; and/or
(ii) the ZP is disrupted enzymatically or mechanically, and a micropipette is used to aspirate the one or more of the embryonic cells from the one or more donor embryos thereby isolating the embryonic cells.

13. (canceled)

14. (canceled)

15. The method of claim 1, wherein the embryonic cells are cultured in the presence of one or more factors capable of promoting embryogenesis and/or totipotency.

16. (canceled)

17. The method of claim 1, wherein the donor embryos are from a mammalian species, optionally wherein the mammalian species is a livestock species, optionally wherein the livestock species is a bovine species.

18. (canceled)

19. (canceled)

20. The method of claim 1, wherein one or more donor embryos at step is/are:
(i) produced by in vivo fertilisation;
(ii) produced by in vitro fertilisation (IVF);
(iii) fresh; and/or
(iv) cryopreserved.

21. (canceled)

22. (canceled)

23. (canceled)

24. The method of claim 1, wherein:
(i) the method comprises selecting the one or more donor embryos prior to step (i) on the basis of one or more genetic screening criteria, genetic diagnoses and/or one or more morphological criteria;
(ii) the one or more donor embryos have been genetically modified; and/or
(iii) the one or more donor embryos comprise a unique genetic tag or identifier for traceability of the embryos produced therefrom and/or animals produced from said embryos.

25. (canceled)

26. (canceled)

27. The method of claim 1, wherein:
(i) the plurality of embryos produced are expanded in vitro to form blastocysts;
(ii) one or more of the embryos produced by the method are harvested and stored in an embryo holding media, optionally at 4° C.; and/or
(iii) one or more of the embryos produced by the method are harvested and cryopreserved.

28. (canceled)

29. (canceled)

30. (canceled)

31. (canceled)

32. (canceled)

33. (canceled)

34. A method of breeding an animal, comprising:
(i) transferring one or more embryos produced by the method of claim 1 to an oviduct(s) of one of more recipient females to establish a pregnancy in the recipient female; and
(ii) producing the animal from a pregnant recipient female by parturition.

35. The method of claim 34, wherein the animal is a mammalian species, optionally wherein the mammalian species is a livestock species, optionally wherein the livestock species is a bovine species.

36. (canceled)

37. (canceled)

38. The method of claim 6, wherein separation of the one or more embryonic cells from the one or more donor embryos is achieved by:
(i) splitting the donor embryo into a plurality of portions, each portion comprising one or more embryonic cells; or
(ii) disrupting a zona pellucida (ZP), and isolating the one or more of the embryonic cells from the one or more donor embryos.

39. The method of claim 38, wherein:
(i) splitting the one or more donor embryos is performed using a microsurgical blade, or laser; and/or
(ii) the ZP is disrupted enzymatically or mechanically, and a micropipette is used to aspirate the one or more of the embryonic cells from the one or more donor embryos thereby isolating the embryonic cells.

40. The method of claim 6, wherein the embryonic cells are cultured in the presence of one or more factors capable of promoting embryogenesis and/or totipotency.

41. The method of claim 6, wherein the donor embryos are from a mammalian species, optionally wherein the mammalian species is a livestock species, optionally wherein the livestock species is a bovine species.

42. The method of claim 6, wherein one or more donor embryos at step (i) is/are:
(i) produced by in vivo fertilisation;
(ii) produced by in vitro fertilisation (IVF);
(iii) fresh; and/or
(iv) cryopreserved.

43. The method of claim 6, wherein:
(i) the method comprises selecting the one or more donor embryos prior to step (i) on the basis of one or more genetic screening criteria, genetic diagnoses and/or one or more morphological criteria;
(ii) the one or more donor embryos have been genetically modified; and/or
(iii) the one or more donor embryos comprise a unique genetic tag or identifier for traceability of the embryos produced therefrom and/or animals produced from said embryos.

44. The method of claim 6, wherein:
the plurality of embryos produced are expanded in vitro to form blastocysts;
one or more of the embryos produced by the method are harvested and stored in an embryo holding media, and/or
one or more of the embryos produced by the method are harvested and cryopreserved.
